(19) United States
(12) Patent Application Publication
(10) Pub. No.: US 2018/0177715 A1
(43) Pub. Date: Jun. 28, 2018

(54) NATURAL BASE TEETH WHITENING GEL CONTAINING NO PEROXYDE

(71) Applicant: Stephane BOCHENEK, Yerres (FR)

(72) Inventor: Stephane BOCHENEK, Yerres (FR)

(21) Appl. No.: 15/820,233

(22) Filed: Nov. 21, 2017

(30) Foreign Application Priority Data
Dec. 28, 2016 (EP) 16207176.5

(51) Int. Cl. A61K 8/9789; A61Q 11/00; A61K 8/04; A61K 8/19; A61K 8/60; A61K 8/73; A61K 8/34

(52) U.S. Cl. CPC A61K 8/9789; A61Q 11/00; A61K 8/042; A61K 2800/30; A61K 8/604; A61K 8/73; A61K 8/345; A61K 8/19

(57) ABSTRACT

The invention relates to a teeth whitening gel containing no peroxidizing agent or petrochemical by-products, and comprising a combination of a whitening agent consisting of sodium or potassium bicarbonate, with one or more humectant polyol(s), one or more gelling polyoside(s), one or more emulsifying alkyl polyglucoside(s), a bactericidal or bacteriostatic plant extract consisting of one or more essential oil(s) and/or plant extract(s), and a method for preparing said gel.

The invention also relates to a cosmetic method for whitening teeth and a teeth whitening kit using said gel.

The present invention relates to a teeth whitening gel containing no peroxide or petrochemical by-product compounds.

Teeth whitening aims at lightening the colour of teeth, for aesthetic purposes. Defective teeth colour can have a natural or psychological origin (for instance resulting from the ageing of teeth), or originate from exogenic factors (for instance tetracycline taken during the formation of teeth, high consumption of coffee, tea or heavy smoking, etc.).

Various types of teeth whitening treatments are now available. They are mainly based on the utilization of peroxide compounds (specifically hydrogen peroxide or carbamide peroxide) as whitening agents. Such peroxide whitening agents have, in addition to a good decolourizing efficiency on the pigments teeth stains consist of, a remarkable bactericidal action, which makes it possible to fight bacteria in the dental plaque.

Some of these treatments are executed at the dentist's office, and require one or more appointment(s). A tailor-made tray fitting the dental arch, filled with a whitening product with a high concentration in whitening agent is applied to the jaw to be treated for an application time of the order of a few dozens of minutes, which may vary according to the initial colour of the teeth. If need be, the lightening process may be activated by using a laser lamp. Such treatments at the dentist's office may be replaced or completed by an ambulatory treatment prescribed by the dentist, which uses the same trays, and a less concentrated whitening product. Trays are applied every day for a time which varies from a few minutes to one hour a day, until the desired lightening is reached.

In addition to the treatments which have to be executed under a dentist's supervision, home treatments also exist, which are sold without a prescription as ready-to-use kits. Like the other ones, such treatments use peroxide-based whitening agents, but the concentration thereof is much lower.

They can take several forms, and combine a whitening composition with an applicator device. The following can be cited:
brush- or pen-shaped applicators: these enable a precise application of the whitening product, for a limited location on a few teeth;
self-adhesive strips coated with a whitening composition, which can be placed on the teeth to be treated;
preformed dental trays, which may be filled with whitening compositions.

In general, teeth whitening compositions are gels which make an extended contact with the teeth enamel possible, and contain gelling agents which give same the desired viscosity. They also contain humectants which enable a good moistening of the gums and an efficient transfer of the active agents onto the teeth surfaces, and emulsifying agents which provide the wettability of the gel on the surfaces of the teeth and make the elimination of food debris and pigments inlaid in the enamel easier.

In spite of the whitening efficiency and the bactericidal properties of the peroxide whitening agents, the utilization thereof raises several problems. As a matter of fact, it has been reported that peroxides could induce an increase in the teeth sensitivity in some persons, as well as gingival irritation. The risk of such side effects is increased with treatments sold without a prescription to be used at home, since checking whether the recommended instructions for use have been complied with is difficult. On the other hand, this method is aggressive and demands remineralization after the treatment, at the risk of obtaining the reverse of the desired effect: as a matter of fact “porous” teeth will very quickly absorb colouring products (tea, coffee, tobacco, wine, fruit, . . . ). Such drawbacks have led law makers, in many countries, to strictly limit the maximum authorized concentration in hydrogen peroxide in the teeth whitening compositions intended for consumers; such maximum authorized concentration is for instance 0.1% in France.

Besides, most of the teeth whitening gels currently sold use carbomers as texturizing agents intended to control the viscosity thereof. Such carbomers are acrylic acid hydrophilic synthetic polymers. They easily give the appearance of an aqueous gel and are thus used as thickening agents in the cosmetic industry. Triethanolamine or aminomethyl propanol makes it possible to neutralize same and to stabilize and control the gel viscosity.

Although they are not considered as toxic for cosmetic use, the petrochemical origin of such compounds, the very low biodegradability and ability thereof to remobilize calcium and magnesium ions are strongly criticized. Additionally, they are not on the list of authorized food additives. Now, when using a teeth whitening gel, a significant part of the product may be swallowed.

The present invention aims at obtaining new natural base teeth whitening compositions, which make it possible to prevent problems related to the utilization of peroxide derivatives and petrochemical by-products, while keeping the instructions for use and preserving the whitening properties of the prior art compositions.

For this purpose, the present invention provides for teeth whitening gels containing sodium or potassium bicarbonate, as the single whitening agent, which ensures whitening through a chemical interaction with anthocyanin or polyphenol deposits fixed on the enamel or the dental plaque. Such whitening agent is associated with at least one plant extract and/or at least one plant essential oil with bactericidal or bacteriostatic characteristics.

The integration of the plant extract and/or the essential oil has raised the problem of their solubilization. Such solubilization is usually carried out by a premixture with a highly ethoxylated castor oil derivative (for instance ethoxylated hydrogenated castor oil containing 40 units of ethylene oxide). Now ethoxylation is considered as a polluting process, liable to generate, upon the implementation thereof, 1,4-dioxane, a contaminant classified as potentially carcinogenic. Therefore, this solution could not be chosen.

During further researches, the inventors noted that non ionic surface-active compounds of the alkyl polyglucoside grade surprisingly had water-solubilizing properties close to those of ethoxylated castor oil, and made it possible not to use same.

Alkyl polyglucosides additionally showed high washing power which made it possible to eliminate coloured deposits on teeth enamel, without any irritation of the gingival mucosa, and enabled the utilization thereof as single emulsifiers in the compositions according to the invention.

In order to avoid the presence of petrochemical by-products, the only gelling agents used in the teeth whitening gels of the present invention are polyosides, and the only humectants used are polyols.

The object of the present invention is thus a teeth whitening gel comprising:

a whitening agent consisting of sodium or potassium bicarbonate;
an emulsifying agent consisting of one or more alkyl polyglucoside(s);
a gelling agent consisting of one or more polyoside(s);
a humectant consisting of one or more polyol(s);
a bactericidal or bacteriostatic agent consisting of at least one plant extract and/or at least one plant essential oil.

The gelling polyosides used in the compositions of the invention have a vegetal or bacterial origin. They may be natural polyosides, or modified polyosides or semi-synthetic polyosides derived from the previous ones.

Polyosides having a plant origin which can be used within the scope of the present invention may originate from algae. Alginates, carrageenans and agar-agar can be cited.

Alginates are polysaccharides extracted from brown algae, specifically fucales and laminariales.

Carrageenans are polysaccharides obtained from red algae, specifically Solieriacae, Gigartinacae, Furcellariacae, Hypneacae, Rhabdoniacae and Rhodophyllidacae. Agar-agar is extracted from the cell wall of some species of red algae, specifically Gelidiacae and Gracilariacae. Purified agarose from agar agar can more particularly be used within the scope of the present invention.

Other plant polyosides which can be used within the scope of the present invention are obtained from terrestrial plants. These can be plant gums (for instance locust bean gum, tara gum, guar gum, Arabic gum, gum ghatti), pectines, starch, and semi-synthetic cellulose or starch derivatives.

Locust bean gum, guar gum and tara gum are extracted from Ceratonia siliqua, Cyamopsis tetragonoloba and Caesalpinia spinosa seeds respectively.

Arabic gum and gum ghatti are produced from exsudations from notches in the trunks of some species of locust trees (specifically Acacia Senegal), for Arabic gum, and Anogeissus latifolia for gum ghatti.

Pectines are mainly produced from fruit plant walls (apples, citrus fruit, etc.) which are by-products of the fruit juice industry. Starch is produced from the parenchyma of miscellaneous plants (corn, manioc, potatoes, etc).

Semi-synthetic or modified derivatives of plant polyosides which may be used within the scope of the present invention are, for instance, carboxymethylcellulose, hydroxyethylcellulose, hydroyethyl-methyl-cellulose, modified starch, etc. . . . .

Bacterial polyosides which can be used in the compositions of the present invention are specifically gellan gum produced by Sphingomonas elodea and xanthan gum produced by Xanthomonas campestris.

Depending on the application mode considered for the teeth whitening gel, the texture and viscosity thereof can be adjusted, specifically through the nature of and concentration in gelling agents and the solution pH, in order to obtain an easy dispensing from the storing device as well as sufficient hold on the teeth surfaces. A combination of both or several gelling polyosides can advantageously be considered. Viscosity is generally considered as optimum between 40,000 and 150,000 mPa·s.

The humectant used in the compositions of the invention consists of a polyol or a polyol mixture.

For instance, monosaccharides can be cited, such as: glycerol, sorbitol, erythritol, pentaerythritol, xylitol, mannitol, arabitol, etc . . . , and disaccharides such as maltitol and isomaltitol or non cariogenic hemiacetalysed oses (cyclized sugar), fructose, galactose, mannose, etc. . . . and diholosides, such as lactose or maltose, etc. . . . .

In a preferred composition, glycerol, sorbitol and xylitol will be associated, with the latter having, on the one hand, a known effect of mouth freshener, and on the other hand inhibiting the production of acids in the dental plaque and reducing adherence and amount of the plaque, which makes it easier to remove when brushing teeth.

The teeth whitening gel according to the invention further comprises a mixture of plant extracts obtained from plants having bactericidal or bacteriostatic properties which thus replaces the bactericidal action of peroxidizing agents. Such extracts ensure a natural preservation of the product, and have an inhibiting effect on the development of the dental plaque.

These may be, for instance, one or more essential oil(s) or hydro-alcoholic or hydro-glycolic extract(s), the bactericidal or bacteriostatic properties of which are known and which can be used in dental hygiene products.

For information, the following essential oils or extracts can be cited: Rosewood, Cajuput, Cinnamon rind, Lemon, Lime, Madagascar Citronella, Copaiba, Turmeric, Eucalyptus Globulus, Eucalyptus Radiata, Geranium, Clove, Cinnamomum Camphora, Bay tree, Lavender Aspic, Fine Lavender, Manuka, Thujanol Marjoram, Bergamot Mint, Peppermint, Spearmint, Niaouli, Oregano, Palmarosa, Norway Pine, Hemlock, Ravintsara, Balsam Fir, Silver White Fir, Siberian Fir, Savory, Melaleucea Alternifolia (Tea plant), Linalol Thyme, Borneol Satureoide Thyme, etc. . . . .

Alkyl glucosides which are used as emulsifying agents in the compositions of the invention are non ionic surfactants of vegetable origin, obtained from starch and fatty acids.

Preferred alkyl polyglucosides are defined by the following formula:

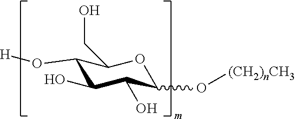

where n is an integer between 7 and 17, preferably between 9 and 15, and m (number of glucose units) is an integer between 1 and 5.

As non restrictive examples of such family of compounds, the following ones can be cited: Plantacare 818 UP produced by the BASF company (cocoyl glucoside), Plantacare 810 produced by the BASF company (caprilyl/capryl glucoside), Triton CG-110 produced by the Dow company, etc. . . . .

The composition which is the subject of the invention may also comprise vegetal extracts or essential oils which have beneficial effects on the gingival mucosa, such as softening, anti-inflammatory and/or antioxidant properties. Such fraction of plant extracts comprises, for instance, a mixture of aloe leaf extract, pomegranate extract and chamomile flower extract.

In a preferred composition, a teeth whitening gel according to the invention may further comprise a plant extract obtained from Salvadora Persica (also called miswak or siwak). Such extract from a shrub of the salvadoraceae family, originating from the Middle-East, is a bacterial inhibitor preventing bacteria from building a dental plaque and makes it possible to prevent oral bacterial halitosis. Besides, it contains thiourea and carbamide sulphur derivatives, as well as thiocarbamide and carbamide derivative alkaloids which make Salvadora Persica extremely efficient as regards teeth whitening. Such extracts are generally in the form of non water-soluble powder or aqueous suspension; thus, they must be incorporated into compositions having a viscosity above 5,000 mPa·s or with a remarkable threshold effect, so that the suspension can be stable; the concentration thereof generally remains less than 0.5% not to significantly affect the gel aesthetics.

A teeth whitening gel according to the invention will also comprise one or more flavouring agent(s), which make(s) its use more pleasant for the consumer. This may be any flavour used in dental hygiene products. It may also comprise one or more colouring agent(s), the nature of which will depend on the colour desired for the preparation.

A teeth whitening gel according to the invention will usually comprise:
from 15 to 50%, preferably from 20 to 35% of an aqueous solution containing 9% of sodium bicarbonate;
from 40 to 70% by weight of a humectant; if such humectant is a mixture of glycerol, sorbitol and xylitol, such mixture will advantageously comprise from 98.8 to 99.8% by weight of glycerol, from 0.2 to 1% by weight of sorbitol, and from 0.05 to 0.2% by weight of xylitol.
from 0.5 to 5% by weight of gelling polysaccharide, preferably comprising from 1 to 1.5% by weight of xanthan or gellan gum, and from 0.1 to 0.5% by weight of carrageenan;
from 0.05 to 0.5% by weight of one or more alkyl polyglucoside(s), characterized in that the alkyl chain thereof comprises from 8 to 18 atoms of carbon, and the glucose chain thereof comprises from 1 to 5 glucose units.
from 0.05 to 0.5% by weight of essential oil(s) or hydro-alcoholic or hydro-glycolic extract(s) of plants, the bactericidal or bacteriostatic properties are known and which can be used in dental hygiene products
the 100% balance of ionized water, if need be.

When flavouring and/or colouring agents are used, the amount thereof will mainly depend on their nature, as well as on the intensity of taste and/or colour which is desired for the final product. For example, if peppermint oil is used for flavouring purposes, it will be used in an amount of 0.05 to 0.2% by weight of the final product.

The object of the present invention also is a method for preparing a teeth whitening gel according to the invention. Such method comprises the following steps:

a. preparing the polyol or the mixture of polyols used as the humectant;

b. incorporating the mixture of gelling polysaccharides into said humectant, with constant stirring until completely dispersed and a transparent and homogeneous gel is formed;

c. keeping the mixture obtained in step b) intermittently stirred for 12 to 48 hours;

d. adding sodium bicarbonate in a water solution previously pre-heated to a temperature under 60° C., with constant stirring until a transparent and homogeneous gel is obtained;

e. agitation adding a premixture consisting of alkyl polyglucoside(s) of vegetable origin slightly heated (35-45° C.) and homogenized with the bactericidal or bacteriostatic essential oil(s) plant extract(s); with said mixture being, for instance, in an alkyl polyglucoside/plant extract ratio of the order of 1:1 to 4:1.

If the teeth whitening gel contains another plant extract, such extract will be added, while stirring, to the gel obtained upon completion of step e), and stirring will go on until it is completely incorporated.

If it is desired to add flavouring and/or colouring agents, these will be added, still while stirring, after the plant extract, and stirring will go on until they are completely incorporated in the mixture.

Another object of the present invention is also an aesthetic method for whitening teeth, characterized in that it comprises applying onto teeth, the aspect of which has to be improved, a teeth whitening gel according to the invention.

The teeth whitening gel according to the invention will generally be used every day or twice-daily, for at least 5 minutes, and preferably from 10 to 60 minutes. However, the frequency of application, as well as the duration thereof, may be increased or reduced if need be. The applications may go on until the desired degree of lightening is reached. Because of the absence of peroxides, an extended treatment entails no risk of teeth being sensitized or the gingival mucosa being irritated.

Another object of the invention is also a teeth whitening kit, comprising a teeth whitening gel according to the invention, and a device for applying said gel.

The applicator device may be brush- or pen-shaped or a stick with a foam tip; it may also be a self-adhesive strip coated with the whitening gel according to the invention or a preformed dental tray, which may be filled with the whitening gel.

The present invention will be better understood when reading the additional description hereunder, which refers to a non restrictive example illustrating the preparation of a teeth whitening gel according to the invention.

### EXAMPLE

Ingredients:

The ingredients used for preparing an exemplary gel formula according to the invention are listed in the table hereunder:

| | Case | Percentage |
|---|---|---|
| Ingredients | number | by weight |
| Glycerol | 56-81-5 | 64.40% |
| Sodium bicarbonate | 144-55-8 | 32% |
| (9% solution) | | |
| Xanthan gum | 11138-66-2 | 2.0% |
| Carrageenan (Chondrus | 9000-07-1 | 0.5% |
| Crispus powder) | | |
| Sorbitol (70% solution) | 50-70-4 | 0.5% |
| Xylitol (50% solution | 87-99-0 | 0.2% |
| in water) | | |
| Cocoyl glucoside | 141464-42-8 | 0.2% |
| Savory essential oil | 8006-90-4 | 0.1% |
| (Satureja Montana) | | |
| Pomegranate extract | 84961-57-9 | 0.05% |
| (Punica Granatum) | | |
| Chamomile extract | 84082-60-0 | 0.05% |
| (Chamomilla Recutita) | | |
| Colouring agent: | 3844-45-9 | (A few drops) |
| CI 42090 | | |
| Colouring agent: | 1934-21-0 | (A few drops) |
| CI 19140 | | |

The ingredients used are produced by SPECTRUM CHEMICAL except for the following ones:

Peppermint oil and pomegranate extract (NATURES FLAVORS)

Chamomile extract (BOTANIC CHOICE)

Colouring agents CI 42090 and CI 19140

As regards the preparation of the gel, glycerol is placed in a stainless steel tank. Stirring is started and the speed thereof is so adjusted as to create a vortex. Cooling fluid is circulated around the tank so as to reduce heat accumulation, so that the temperature of the preparation does not exceed 40° C.

Sorbitol (70% aqueous solution) and xylitol solutions (50% aqueous solution) are added into the vortex, with mixing lasting for at least 10 minutes.

Powder ingredients (xanthan gum and Chondrus Crispus powder) are sprinkled into the vortex and mixing goes on until all powders are completely dispersed.

Mixture is carried out intermittently for 2 days, using the above protocol. A thick gel is formed after swelling of the polymers.

On day 3, sodium bicarbonate solution is added while stirring at mid-speed.

Once the desired consistency is reached, speed of the mixer is so adjusted as to keep the preparation moving, and stirring goes on for 5 to 10 minutes.

The premixture of cocoyl glucoside, preheated to 45° C. and homogenized, and the bactericidal essential oil (for instance savory or thyme essential oil) are then added and stirring goes on for about 20 minutes.

Promegranate extract and chamomile extract are then added to the preparation and mixing goes on for 3 to 5 minutes.

Eventually 7 drops of colouring agent CI 42090 and 1 drop of colouring agent CI 19140 per 5 liters of gel are added and mixing goes on for 10 minutes.

1. A teeth whitening gel comprising:
a whitening agent consisting of sodium or potassium bicarbonate;
an emulsifying agent consisting of one or more alkyl polyglucoside(s);
a gelling agent consisting of one or more polyoside(s);
a humectant consisting of one or more polyol(s);
a bactericidal or bacteriostatic agent consisting of at least one plant extract and/or at least one plant essential oil.

2. A teeth whitening gel according to claim 1, characterized in that:
the whitening agent consists of sodium bicarbonate;
the emulsifying agent comprises at least cocoyl polyglucoside;
the gelling agent comprises at least one carrageenan and xanthan gum;
the humectant comprises at least one polyol selected among glycerol, sorbitol and xylitol or a mixture of at least two of said polyols;
the bactericidal or bacteriostatic agent comprises at least one plant extract selected among: Cinnamon rind, Lemon, Lime, Madagascar Citronella, Eucalyptus Globulus, Clove, Bergamot Mint, Peppermint, Spearmint, Oregano, Savory, Melaleucea Alternifolia, Thyme.

3. A teeth whitening gel according to claim 1, characterized in that it further comprises at least one extract or one or more plant(s) having softening, anti-inflammatory and/or antioxidant properties.

4. A teeth whitening gel according to claim 3, characterized in that said plant extract comprises a mixture of a pomegranate extract and a chamomile flower extract.

5. A teeth whitening gel according to claim 4, characterized in that said plant extract further comprises a suspension or a liquid extract of Salvadora Persica.

6. A teeth whitening gel according to claim 1, characterized in that the humectant is a mixture of glycerol, sorbitol and xylitol.

7. A teeth whitening gel according to claim 1, characterized in that it comprises:
from 15 to 50%, preferably from 20 to 35% of a solution containing 9% of sodium bicarbonate in water;
from 40 to 70% by weight of a humectant;
from 0.5 to 5% by weight of a gelling agent;
from 0.05 to 0.5% by weight of an emulsifying agent;
from 0.05 to 0.5% by weight of a bactericidal or bacteriostatic agent.

8. A teeth whitening gel according to claim 1, characterized in that it further contains one or more flavouring agent(s) and one or more colouring agent(s).

9. A method for preparing a teeth whitening gel according to claim 1, characterized it that it comprises the following steps:
a. preparing the polyol or the mixture of polyols used as the humectant;
b. incorporating the gelling agent into said humectant, with constant stirring until completely dispersed and a transparent and homogeneous gel is formed;
c. keeping the mixture obtained in step b) intermittently stirred for 12 to 48 hours;
d. adding the whitening agent in a water solution previously pre-heated to a temperature under 60° C., with constant stirring until a transparent and homogeneous gel is obtained;
e. agitation adding a premixture consisting of the emulsifying agent slightly heated (30-35° C.) and homogenized with the bactericidal or bacteriostatic plant extract.

10. A method according to claim 9, characterized in that it further comprises:
f. agitation adding the plant(s) extract to the product obtained after step e)
g. optionally agitation adding the flavouring agent(s) and the colouring agent(s).

11. A cosmetic method for whitening teeth, characterized in that it comprises applying a teeth whitening gel according to claim 1 onto teeth, the aspect of which has to be improved.

12. A teeth whitening kit, characterized in that it includes a teeth whitening gel according to claim 1, and a device for applying said gel onto teeth, the aspect of which has to be improved.
